(19) United States
(12) Patent Application Publication
(10) Pub. No.: US 2006/0178623 A1
(43) Pub. Date: Aug. 10, 2006

(54) Needle holder for safety syringe

(76) Inventor: Cheng-Chi Lo, Taipei (TW)

Correspondence Address: BACON & THOMAS, PLLC, 625 SLATERS LANE, FOURTH FLOOR, ALEXANDRIA, VA, 22314, US

(21) Appl. No.: 11/042,057

(22) Filed: Jan. 26, 2005

(51) Int. Cl. A61M 5/00; A61M 5/32

(52) U.S. Cl. 604/110; 604/192

(57) ABSTRACT

The present invention is an improved needle holder for safety syringe, which is a needle holder formed through compounding two materials in one-time, in which rubber convex ring of high coefficient of deformation is formed on the step ring of holder, such that the convex ring can properly compress the ring slot at the front part of barrel. Further, a buckle ring is formed inside the holder, such that the buckle head of plunger locks the buckle slot after injection, and the holder as well as the needle are pulled back into the barrel when the plunger is pulled, thus prevents the needle from exposure.

### BACKGROUND OF THE INVENTION

1) Field of the Invention

The present invention relates to an improved needle holder for safety syringe. More specifically, it relates to the material for locating as well as the structural design of a needle holder for safety syringe, in which the technology of compounding plastic and rubber materials is applied, such that the holder can be properly compressed at the front end of barrel and thus relieves and retracts from the font end of barrel.

2) Description of the Prior Art

Conventional needle holder for safety syringe provides a hook part at the front of plunger. When the plunger is pushed to the end, the hook part hooks the needle holder, and the needle holder along with the needle are retracted back to the barrel while the plunger is pulled back, the contamination caused by careless piercing is thus avoided.

The inventor of the present invention has strived for the development of safety syringe over a long period of time, and he has been granted numerous patents. Since the conventional needle holder and the barrel are manufactured by multi-cavity plastic injection molding, and as the injection environment and condition of different cavities are not necessarily the same, while there generally exist distinctions between multiple molds in mass production, it is difficult to precisely control the sizes of respective needle holder and barrel after injection forming. Consequently, it is hard to match each other, and the manufacturers are upset by the resultant low yield rate.

Therefore, based on the description in a previously submitted application for Taiwan patent and the structure as well as the operation principles of “needle holder for safety syringe”, which was a invention patent with grant number of 194836, the inventor of the present invention herein further improves the two-piece needle holder into a one-piece type. Besides, since the improved holder can be embodied and inlaid into the preceding structure of components, hence additional design of barrel or other component mold are unnecessary.

### SUMMARY OF THE INVENTION

To meet the aforementioned requirements, the main purpose of the improved needle holder for safety syringe according to the present invention provides a holder that the technology of compounding plastic and rubber materials to compound two materials is applied and is formed in one-time process, such that the quantity of components, cost, and manufacturing processes are all reduced. Besides, making convex ring of holder from rubber material of high coefficient deformation, the allowed range of inaccuracy certainly becomes broader, and the yield rate is accordingly raised.

### BRIEF DESCRIPTION OF THE DRAWINGS

FIG. 1 is an exploded view of the present invention.

FIG. 2 is a 90 degree sectional view of the present invention.

FIG. 3 is a vertically sectional view of the present invention.

FIG. 4 shows the action of pushing the plunger forward according to the present invention.

FIG. 5 shows that the needle and the holder are retracted into the barrel according to the present invention.

FIG. 6A is another embodiment of the buckle head according to the present invention.

FIG. 6B is another embodiment of the buckle head according to the present invention.

### DETAILED DESCRIPTION OF THE PREFERRED EMBODIMENTS

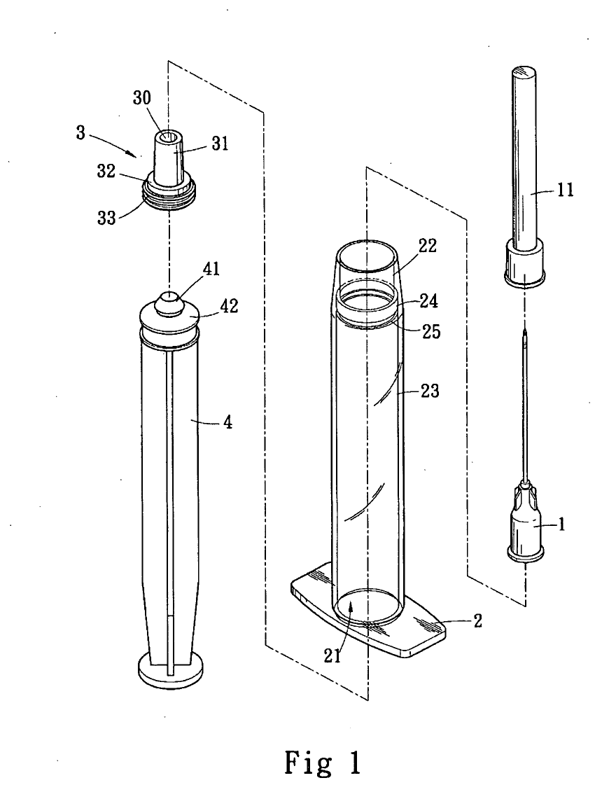

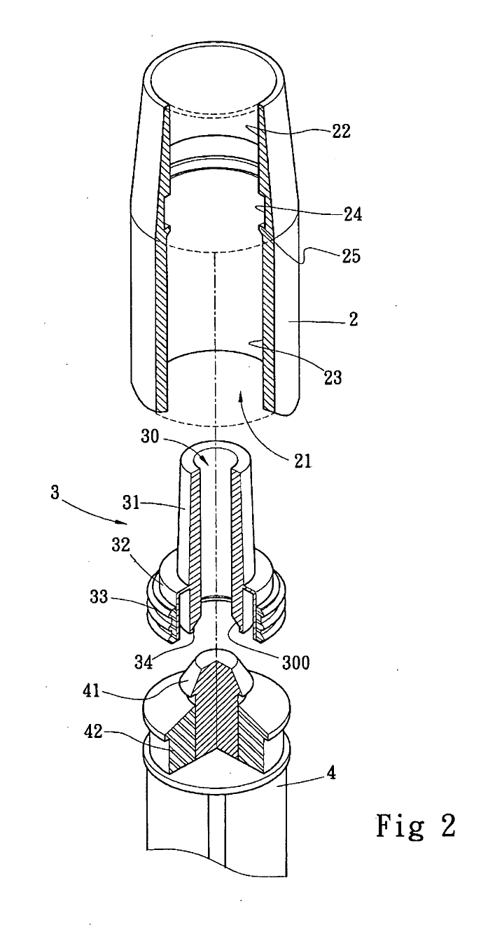

Referring to FIGS. 1 and 2, the safety syringe is composed of a needle cap 11, a needle 1, a barrel 2, a holder 3, and a plunger 4, in which the barrel 2 is a hollow tube and a room 21 is thus formed. A height-drop ring slot 24 of large diameter is provided on the upper side of inner wall 22 of the room 21 at the font end of the barrel 2, while a resist ring 25 is formed in ring slot 24 and is neighbored to the low-inner wall 23, in which the diameter of the low-inner wall 23 is larger than that of the up-inner wall 22 yet smaller than the diameter of ring slot 24. Ring slot 24 is designed for supporting the compressing and buckling applied by convex ring 33 of holder 3.

Further, a needle base 31 is formed on the holder 3 for inserting and fixing needle 1, the lower side of needle base 31 is provided a height-drop to form a step ring 32, and the peripheral of step ring 32 is formed a convex ring 33, in which the convex ring is made from rubber material, and the holder 3 can be formed in one-piece injection molding. As the rubber material has higher coefficient of deformation, the resist ring 25 and ring slot 24 of barrel 2 can endure higher range of inaccuracy between each other, and thus significantly raises the yield rate.

Moreover, a hole 30 is provided at the center of needle base 31 such that medication can be injected into the needle 1. A ramp 300 is provided at the bottom of hole 30 to meet the cone of buckle head 41, in which a buckle ring 34 is provided at the opening edge of the ramp 300 in order to be buckled to the buckle head 41.

Besides, a cone buckle head 41 and an enveloped rubber head 42 are provided at the end part of the plunger 4, in which the buckle head 41 is droppable into the buckle ring 34 of ramp 300, thus constructs a structural design that allow one-way pushing only whereas breaking away is not permitted. Consequently, after pushing the plunger 4 to the end, the buckle head 41 will not break away from the holder 3 once getting jointed with buckle ring 34.

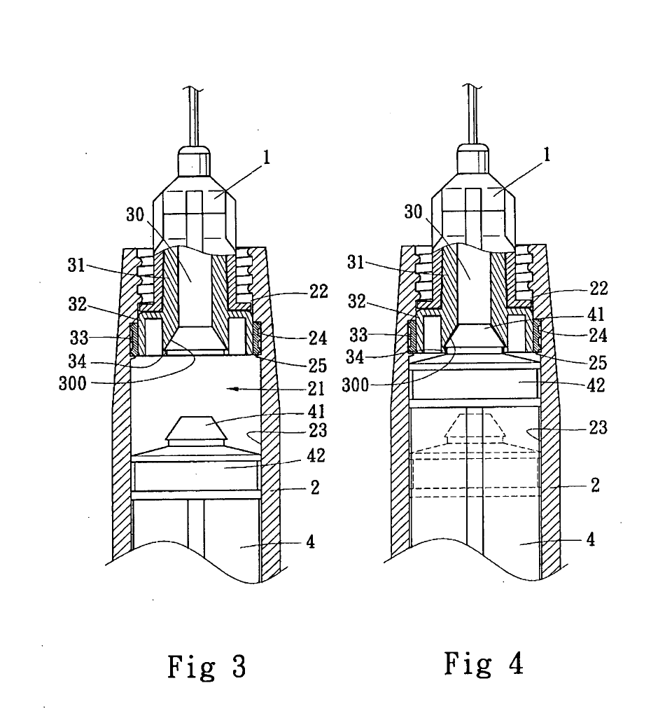

Further, referring to FIGS. 3 and 4, during injection, plunger 4 is pushed forward in the room 21 of barrel 2, once the buckle head 41 drops into buckle ring 34, the unbreakable joint is completed, and the buckle head 41 is attached to the ramp 300 of the hole 30. Such a design not only expects that the medication is injected completely, but as well broaden the allowed error value when cone buckle head 41 contacts ramp 300 and firmly joints to the holder.

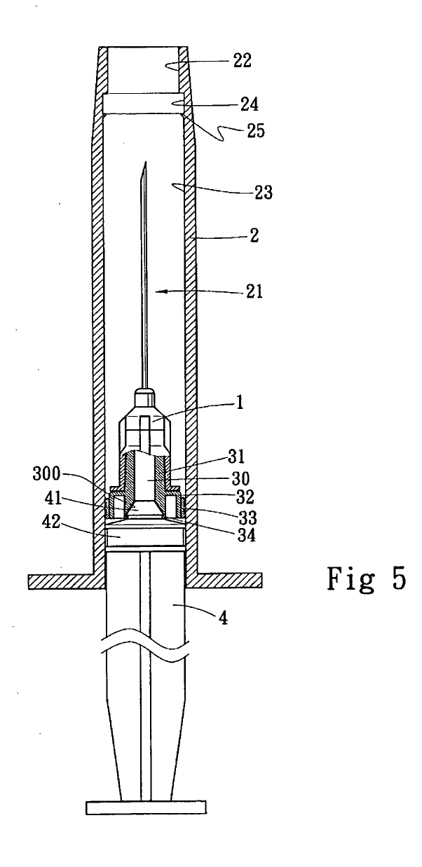

On the other hand, during the period of pulling plunger 4, as the buckle ring 34 is made from rubber material, the margin of allowed deformation is large, and once the force of buckling the buckle ring 34 and the buckle head 41 is greater than the compressing of convex ring 33 to the ring slot 24, the plunger 4 is pulled back, and the convex ring 33 is forced to break away from the resist of ring slot 24 and resist ring 25, the holder 3 and needle 1 are integrally retracted back to the room 21 of barrel 2. The operation of retracting safety needle is thus completed, as shown in FIG. 5.

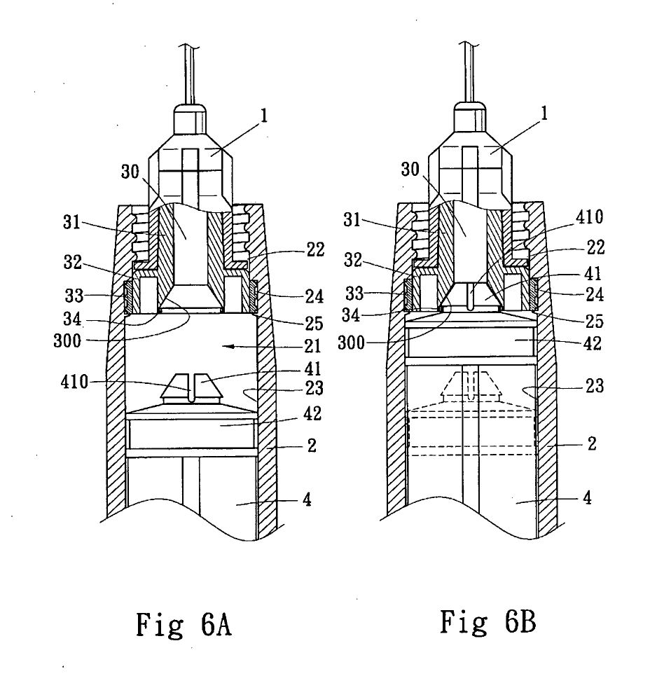

Furthermore, referring to FIGS. 6A and 6B, which show another embodiment of the buckle head according to the present invention. Considering that the force of buckling the buckle head 41 to the buckle ring 34 should be larger than the compressing of convex ring 33 to the ring slot 24 to firmly and safely pull back the holder 3 as well as the needle 1, the size of lock 41 becomes critical, thus this embodiment provide a gap 410 in buckle head 41 such that the largest allowed error value to the diameter of the cone is increased.

As described in the background and main purpose of the present invention, the holder 3 is a critical component of the whole safety syringe. Since the assembly of installing the holder 3 to the barrel 2 significantly influences the yield rate of the whole safety syringe. By way of the technology of compounding plastic and rubber materials and applying the rubber material to embody the convex ring 33 of the holder 3 on account of its high coefficient of deformation, the holder 3 can be compressed properly at the front part of barrel 2, the allowed range of inaccuracy is thus raised, and the yield rate is accordingly raised.

1. An improved needle holder for safety syringe, the safety syringe at least includes: a needle cap, a needle, a barrel, a holder, and a plunger, which mainly applies the technology of compounding rubber and plastic materials to the compressing structure of holder, which features:
A barrel, which is a hollow tube and a room is thus formed, in which a height-drop ring slot of large diameter and a resist ring are provided at the font end of the barrel, where the ring slot is provided to be buckled by the needle base;
a holder, which is formed in one-time injection molding from compounding two materials, in which a needle base and a step ring are formed, where a convex ring is formed around the peripheral of step ring, and a hole is provided at the center of needle base, while a ramp is provided at the bottom of the hole, and a buckle ring is provided at the opening edge of the ramp;
a plunger, which is jointed to the buckle base of the holder, where a buckle head and an enveloped rubber head are provided at the end part;
as the convex ring is formed by rubber material, and the coefficient of deformation for rubber material is large, the allowed range of inaccuracy is broadened, the yield rate is thus significantly raised.

2. An improved needle holder for safety syringe as claimed in claim 1, wherein the buckle head of plunger can drop into buckle ring of holder for buckling and joint, and forms a structure that allows one-way pushing and buckling only whereas breaking away is not permitted.

3. An improved needle holder for safety syringe as claimed in claim 1, wherein the buckle head of plunger can be embodied into a shape of cone or round.

4. An improved needle holder for safety syringe as claimed in claim 1, wherein the buckle head of plunger can provide a gap of minor span at its center to increase the force of buckle between buckle head and the holder.

5. An improved needle holder for safety syringe as claimed in claim 1, wherein the buckle ring can be embodied using rubber material since the high coefficient of deformation for rubber material can significantly raise the yield rate.
